(19) United States
(12) Patent Application Publication
(10) Pub. No.: US 2017/0106107 A1
(43) Pub. Date: Apr. 20, 2017

(54) METHOD FOR PROCESSING COSMETIC PRODUCTS UNDER HIGH HYDROSTATIC PRESSURE, PRODUCTS OBTAINED AND SUITABLE CONTAINER

(71) Applicant: HPBIOTECH, Gradignan (FR)

(72) Inventor: Gerard DEMAZEAU, GRADIGNAN (FR)

(21) Appl. No.: 15/300,621

(22) PCT Filed: Mar. 31, 2015
(86) PCT No.: PCT/FR2015/050833

§ 371 Date: Sep. 29, 2016

(30) Foreign Application Priority Data
Mar. 31, 2014 (FR) 1452776

(51) Int. Cl. A61L 2/02; A61K 8/02; A61Q 19/00

(52) U.S. Cl. CPC A61L 2/02; A61Q 19/00; A61K 8/0216; A61K 2800/30; A61K 2800/805; A61K 2800/10; A61K 2800/87

(57) ABSTRACT

A method for processing a cosmetic product with high hydrostatic pressures by applying a pressure P to the cosmetic product, with a continuous or cyclic application mode MA. The method is performed using at least one, and preferably all, of the following parameters: total application time t of pressure P of between 2 and 60 minutes, a pressure P of between 200 MPa and 600 MPa, a pressure application rate, for a compression VA, of between 1 and 5 QMPa/s, a decompression rate VA_{D} of between 1 and 50 MPa/s, when the application mode is cyclic, a latency time tₗₐₜ applied before the first cycle and/or between certain cycles and/or between each cycle, and an initial temperature of between −10° C. and 40° C. Also, the cosmetic product obtained and a device for implementing. The method can be used to deactivate microorganism(s) in pharmaceutical product or to modify the texture of the same.

### FIELD OF THE INVENTION

This invention relates to a method for treating cosmetic products by high hydrostatic pressure, in particular for inactivating in an irreversible manner the pathogenic microorganisms that are likely to contaminate them while preserving their properties. The objects of the invention are also the products that are obtained as well as a container that is suitable for the treatment of cosmetic products and their preservation after treatment.

### BACKGROUND OF THE INVENTION

The cosmetic products are sensitive products that can be contaminated by certain microorganisms. So as to prevent any health problem, in particular with contact of the skin in the case of a cutaneous use, it is necessary to decontaminate these products. Different techniques have been described for this purpose: thermal techniques such as pasteurization and optionally sterilization, techniques for treatment by ionizing radiation, and more recently other physical techniques such as ultrasound or microwaves. However, all of these methods, in particular through developed energy, can lead to an alteration of the tight structure of treated cosmetic products (destabilization of emulsions, formations of free radicals, etc.) and therefore their inherent properties.

Furthermore, so as to prevent any proliferation of residual microorganisms in cosmetic products treated with the above-mentioned methods or in untreated cosmetic products, it is necessary to resort to the use of agents called “preservatives,” having either a bactericidal effect or effects limiting the microbial development. However, for several years, various regulations have limited the use of some of these preservative additives. In addition, in the case of products with the “Bio” label, these preservative agents of a chemical nature modify the image of neutrality associated with these products.

Also, there is therefore a significant need for a method remedying the drawbacks of the prior methods, making it possible in particular to sterilize a cosmetic product and making possible its preservation without resorting to chemical additives, while preserving its characteristic properties.

It is the objective of this invention that, for responding thereto, proposes a treatment method based on the technology of high hydrostatic pressure (or “High Hydrostatic Pressure” HHP).

The HHP methods are known for the treatment of certain media. In particular, it was shown that the application of HHP on food products can lead to reducing the microbial feedstock (yeasts, molds, bacteria, . . . ) and thereby can make possible an increase in their preservation time (E. RENDUELES et al. “Microbiological Food Safety Assessment of High Hydrostatic Pressure Processing: A Review,” LWT-Food Science and Technology (2011), 44, pp. 1251-1260). However, it is necessary to emphasize that the conditions for inactivation of these microorganisms depend both on the nature of the microorganism (for example, the nature of the strain), and the composition of the medium. In particular, the carbohydrates, the proteins, the lipids and other components (concentrations of salt and of sugars . . . ) can have a protective effect for the microorganisms in relation to high-pressure effects (J. C. CHEFTEL “Review: High Pressure, Microbial Inactivation and Food Preservation,” Food Science and Technology International (1995), 1 (2-3) pp. 75-90, C. GARCIA-GRAELLS et al. “Inactivation of E. coli in Milk by High Hydrostatic Pressure Treatment in Combination with Antimicrobial Peptides,” J. Food Prot. (1999), 62, pp. 1248-1254). In parallel, certain physico-chemical properties of the treated medium can have a strong impact on the sensitivity to the pressure of the microorganisms, in particular the value of the pH and the value of the water activity Aw (J. P. P. M. SMELT “Recent Advances in the Microbiology of High Pressure Processing,” Trends Food Sci. Technol. (1998, 9, pp. 152-158). The inactivation of microorganisms under the effect of the HHP application is therefore a complex phenomenon since it is greatly dependent on the numerous factors that are characteristic of the treated medium.

Thus, microbial inactivation by an HPP method within various cosmetic media that are often rich in lipids (for example, the emulsions of an oily phase in an aqueous phase or of an aqueous phase in an oily phase), or containing polysaccharides, has until now seemed difficult to achieve.

In addition, certain cosmetic products that are fragile in nature, such as emulsions, have a tight structure that can be altered by application of high pressure because of their metastable nature (from a thermodynamic standpoint).

### SUMMARY OF THE INVENTION

Nevertheless, in a surprising way, using the specific combination of particular parameters that act by synergy, the invention consists of a method for HHP treatment of cosmetic products that makes it possible to sterilize them and to inactivate in an irreversible manner the pathogenic microorganisms that can contaminate them without using any preservative-type additive and while preserving their characteristic properties.

In particular, the object of the invention is a method for treating a cosmetic product by high hydrostatic pressure, consisting in applying a pressure P to the cosmetic product, with a continuous or cyclic application mode MA, characterized by a rate VA for application of the pressure P for compression of between 0.5 and 50 MPa·s⁻¹, and at least one of the following parameters, preferably all:
A decompression rate VA_{D} of between 0.5 and 50 MPa·s⁻¹,
A total time t for application of the pressure P of between 2 and 60 minutes,
A pressure P of between 200 MPa and 600 MPa,
An initial temperature of the cosmetic product to be treated of between −10° C. and 40° C.

Advantageously, such a method makes it possible to decontaminate the treated cosmetic products by inactivating in an irreversible manner the pathogenic microorganisms that can contaminate them, while preserving their inherent properties.

The objects of the invention are also the uses of this method in particular for sterilizing cosmetic products, and the cosmetic products that are obtained.

Finally, the object of the invention is also a suitable device for containing products to be treated and products treated by high pressure, in particular cosmetic products.

### BRIEF DESCRIPTION OF THE DRAWINGS

The invention is now described in detail, with the description of the device being established with regard to the accompanying drawings, drawings in which the different figures show:

FIG. 1: Diagrammatic cutaway view of a sealed flexible envelope.

FIG. 2: Diagrammatic cutaway view of an outer container designed to accommodate the flexible envelope.

FIG. 3: Diagrammatic view of a distributor plug.

FIG. 4: Diagrammatic view of the elements of FIGS. 1-3, assembled.

### DETAILED DESCRIPTION OF THE INVENTION

The object of the invention is therefore a method for treating a cosmetic product by high hydrostatic pressure, consisting in applying to the cosmetic product a pressure P, with a continuous or cyclic application mode MA. Preferably, the application mode is cyclic.

In terms of the invention, “cosmetic products” are defined as both:
The cosmetic compositions, finished products designed to be applied on the skin, the mucous membranes, or the integuments, and
The components of these compositions, in particular the cosmetic active ingredients and the additives or vehicles, such as, for example, plant extracts or floral waters.

The “cosmetic products” can come in any form suited to treatment by HHP, i.e., in any form able to conduct pressure, in particular creams, ointments, serums, lotions, products based on floral waters, emulsions, and certain powders having a surface treatment and a grain size that are suitable and known to one skilled in the art so as to reduce the effects of compacting.

The method of high hydrostatic pressure according to the invention is characterized by a certain number of parameters, in particular:
The value of the pressure P,
The rate of application of pressure for the compression VA,
The decompression rate VA_{D}, VA and VA_{D} able to be identical or different,
The mode of application of the pressure MA that shows the way in which the former is applied during the treatment: either in a continuous manner with the same value of pressure during the entire treatment time once the desired value is reached, or in a cyclical manner,
The initial temperature Ti of the cosmetic product to be treated,
The total time t for application of the pressure P for the method.

The pressure P of the method according to the invention is between 200 MPa and 600 MPa.

The rate VA of application of the pressure P for the compression is between 0.5 and 50 MPa·s⁻¹, and preferably the decompression rate VA_{D} is between 0.5 and 50 MPa·s⁻¹.

In a preferred way, the method has a total time t of application of the pressure P of between 2 and 60 minutes.

The initial temperature Ti of the cosmetic product to be treated is preferably between −10° C. and 40° C.

According to a particularly suitable embodiment, the object of the invention is a method for treating a cosmetic product by high hydrostatic pressure, consisting in applying to the cosmetic product a pressure P, with a continuous or cyclic application mode MA, preferably cyclic, characterized by at least all of the following parameters:
A total time t of application of the pressure P of between 2 and 60 minutes,
A pressure P of between 200 MPa and 600 MPa,
A rate VA for application of the pressure P for compression of between 0.5 and 50 MPa·s⁻¹,
A decompression rate VA_{D} of between 0.5 and 50 MPa·s⁻¹,
An initial temperature Ti of the cosmetic product to be treated of between 10° C. and 40° C.

In the case of a cyclic application, the method consists of a series of cycles. A cycle is described by a rise in pressure with the rate VA, a holding of the pressure to the value P for a time t_{c}<t), and then a drop in pressure with a rate VA_{D}:
Either to the ambient pressure Pₒ,
Or to an intermediate pressure Pᵢₙ between the ambient pressure Pₒ and the highest of the pressure P applied during said two cycles.

The time t_{c} of application of the pressure P of each cycle is preferably between 2 and 20 minutes, preferably between 3 and 15 minutes. Either all of the cycles have an identical time t_{c}, or at least two of the cycles have a different time t_{c}.

Likewise, the pressure P can be identical for all of the cycles, or, for at least two of the cycles, the applied pressure P is different.

The rate of compression VA or of decompression VA_{D} can be identical for all of the cycles, or at least two of the cycles have a rate of compression VA and/or of decompression VA_{D} that is different. In certain cases, for particular products, it is necessary to apply a specific compression or decompression profile characterized in that the rate VA or VA_{D} can vary.

Actually, each cycle has a compression and decompression profile. The compression or decompression profile corresponds to the general appearance of the application of pressure during the rise or fall. Thus, the compression and the decompression can be done in a monotonous manner (at a constant rate or with variable rates) or with one or more pressure stages. Each stage is carried out by applying an intermediate constant pressure of a value located between the lowest pressure of the cycle and the highest pressure of the cycle. Reaching this or these stage(s) can be done at a constant rate or at variable rates. Before and after each stage, the application rate can be identical or different.

Also, according to an embodiment, for at least one cycle, the compression rate VA and/or the decompression rate VA_{D} vary(ies) until reaching the pressure P. Either all of the cycles have the same compression and/or decompression profile or at least two cycles have a compression and/or decompression profile that is/are different.

Furthermore, for the same cycle, the compression profile and the decompression profile can be identical or different, i.e., for the same cycle, the rates VA and VA_{D} can be identical or different.

A latency time tₗₐₜ can be applied before the first cycle and/or between certain cycles and/or between each cycle: either at the ambient pressure Pₒ, or at the intermediate pressure Pᵢₙ. This latency time tₗₐₜ is between 2 and 20 minutes. According to a particular embodiment, the method comprises at least three cycles, and at least one latency time tₗₐₜ. If there are at least two latency times tₗₐₜ, these latency times can be identical or different.

The total time t for application of the pressure P for the method is between 2 and 60 minutes. In the case of a cyclic application MA, it is dependent upon the number of cycles and the time of each cycle t_{c} at the treatment pressure P, of the duration of the latency time tₗₐₜ, of rates VA and VA_{D}.

The number of cycles depends on the barosensitivity of the microorganisms to be inactivated, and the treated cosmetic medium therefore determines the total time t of holding under high pressure. This number of cycles cannot exceed 15 cycles.

The initial temperature Ti of the medium is between −10° C. and 40° C. This temperature can be the ambient temperature (20 to 25° C.) or a lower temperature, and even a negative temperature or else a higher temperature. The low temperatures (less than 10° C.) and negative temperatures are well suited to very fragile cosmetic products (such as, for example, those that contain thermolabile active ingredients), because they make it possible to preserve specific characteristics, and the higher temperatures of between 30 and 40° C. make it possible to ensure a very good inactivation of spores when the cosmetic product contains them.

The compression rate, i.e., the rate of the rise in pressure, is between 0.5 and 50 MPa·s⁻¹, in particular between 1 and 50 MPa·s⁻¹. Preferably, it comprises between 1 and 10 MPa·s⁻¹, even more preferably between 1 and 6.6 MPa·s⁻¹. This rate can be constant or variable until reaching the desired pressure. Likewise, for two different cycles, the compression rate can be identical or different.

The decompression rate, i.e., the rate of pressure decrease, is between 0.5 and 50 MPa·s⁻¹, in particular between 1 and 50 MPa·s⁻¹. Preferably, it is between 1 and 10 MPa·s⁻¹ and even more preferably between 1 and 6.6 MPa·s⁻¹. This rate can be constant or variable until reaching the desired pressure. Likewise, for two different cycles, the decompression rate can be identical or different.

For the same cycle, or when the application mode of the pressure MA is continuous, the compression and decompression rates can be identical or different.

The method is implemented using “high-pressure” devices that are suited to the conditions of the invention.

So as to prevent any subsequent contamination after the HHP treatment, the cosmetic product to be treated is to be pre-packaged in its final packaging or a part of its final packaging. This packaging should:
Be entirely or partly deformable to be able to transmit the pressure, i.e., so that the application of pressure can compensate for the compressibility of the treated cosmetic medium,
Not be altered by the application of the HHP treatment, i.e., it must have mechanical strength, chemical integrity, and suitable barrier properties,
Preferably not cause contamination of the product during its use by the consumer, such as in particular “single-dose”-type systems or any system preventing re-contamination by return of a little product or air into the packaging reservoir.

For example, it may involve a packaging made of rigid material, such as glass, plastic, or any other appropriate material to be used as a container, closed by a deformable lid or having a deformable part.

The thus packaged cosmetic products are then placed in a piece of high-pressure equipment, and then this piece of equipment, called “high-pressure chamber,” is closed by at least one seal ensuring sealing. There is generally only a single seal when the “high-pressure” chamber is in the vertical position, and there are generally two seals (one at each end) when the chamber is in the horizontal position.

Pressure is transmitted to the samples by a liquid that does not alter the packaging during the treatment by the HHP method according to the invention. In some cases, a gas can be used so as to preserve the packaging (in particular of the airless type).

The increase in pressure can be carried out either using one or more pumps (in this case, the volume of the “high-pressure” chamber is generally constant) or by reduction of the volume of the chamber.

The different parameters characterizing the HHP treatment to be applied are acquired on a robot or on a computer (depending on the system of data transmission to the piece of equipment). Once the treatment has ended, the “high-pressure” chamber is opened, and the samples are recovered. In the case where the cosmetic product has been treated directly in its final packaging (marketing packaging), it can be either repackaged in an outer packaging, or stored or sent to clients. In the case where the preparation is part of the final packaging, the former is then associated with various elements of said final packaging.

It is possible to treat the outer surface of the packaging or to select the outer layer of this packaging so as to avoid any wetting effect by the liquid that is used as a pressure-transmitting medium.

According to a particularly suitable variant, the cosmetic product is packaged in a specific device, which makes it possible both to apply the method according to the invention and to preserve the product after treatment.

This device, making it possible to apply a high-pressure method, in particular the method according to the invention, to a product, in particular to a cosmetic product, can take the following form.

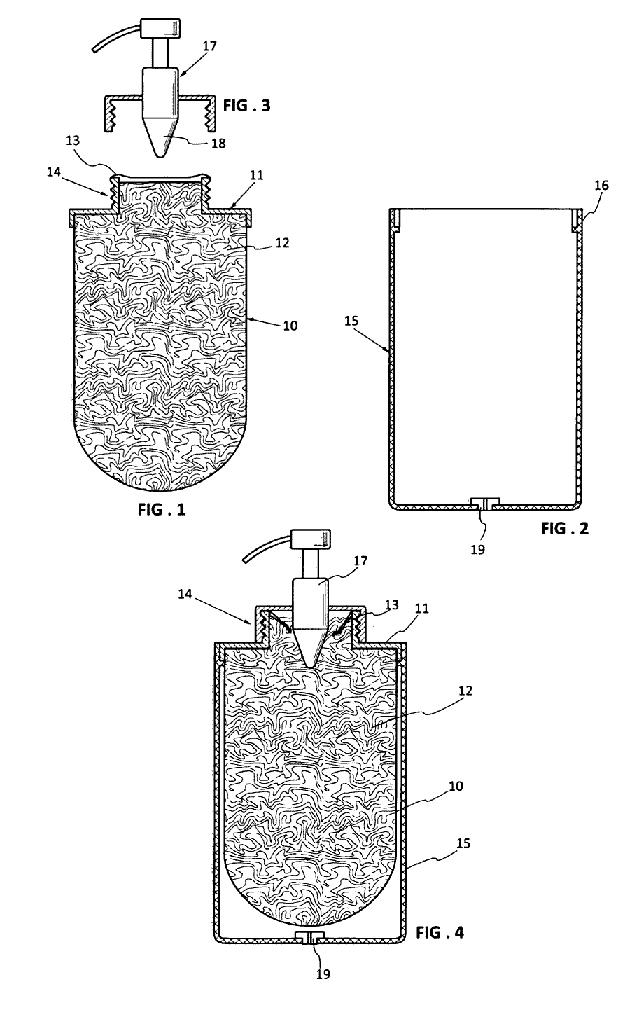

FIG. 1 showed a flexible envelope-reservoir 10 with an opening 11 at the top. A product 12, in this case a cosmetic product, is introduced into this envelope by any known means, such as a filling spout, not shown.

After filling by the cosmetic product, this flexible envelope-reservoir 10 is hermetically sealed by a lid 13 in such a way as to make said envelope-reservoir airtight.

The flexible envelope-reservoir is either attached or comprises a head ring 14.

This envelope-reservoir of the cosmetic product is subjected to the treatment method according to the invention, which has as its object to destroy in a certain way all of the microorganisms that are optionally present in said product and in said envelope-reservoir 10.

Once treated, the flexible envelope-reservoir 10 is placed in an outer container 15, forming a rigid peripheral shell that can bear informational notices, the communication or any other writing or illustration that would prove desirable (FIG. 2).

The ring 14 that is associated with the envelope-reservoir is adapted to the outer container 15, in particular by screwing or gripping (FIG. 3).

The envelope-reservoir 10 is provided with an opening 11 that is equipped with threading or a gripping means for accommodating a plug 17 with a distributor pump, of the “airless” type, i.e., its operation makes it possible to distribute the product without thereby compensating for the volume of product distributed by an air intake of the balancing volume. The plug-distributor pump 17 comprises a canula 18 for intake of the product into the envelope-reservoir that can perforate the flexible lid 13, during the mounting of the head, and can enter into contact with the contents of this flexible envelope-reservoir (FIG. 3).

The outer container comprises an air intake 19 because if the balancing air is not introduced into the rigid outer container 15 to offset the delivery of the product, it is necessary to balance the reduction of the collapsed volume via an air intake into said outer container 15. This air is not in contact with the contents of the envelope-reservoir, which therefore does not pose any re-contamination problem. The air intake can be a simple hole optionally provided with a flap or a valve, if it is desired that access to the volume between the envelope and the outer container be limited only to air.

The plug-distributor pump 17 can be made of a polymer material with biocide properties or may have been treated in advance and removed from a suitable packaging immediately before its installation in such a way as to prevent the introduction of microorganisms.

This device is particularly suitable for containing the cosmetic products during the high hydrostatic pressure treatment according to the invention, and for the preservation of the products once treated.

Advantageously, the combination of the different characteristics of the method according to the invention leads to the destruction in an irreversible manner of the pathogenic microorganisms that are likely to contaminate the treated cosmetic product.

The microbial agents encountered in the cosmetic products are of various types. Various reports or documents specify the nature of these contaminants according to the basic ingredients used, according to the type of cosmetic product and its manufacture, based on the method of use (R. M. BAIRD “Microbial Contamination of Cosmetics Products,” J. Soc. Cosmet. Chem. (1977), 28, pp. 17-20, R. CAMPANA et al. “Microbiological Study of Cosmetic Products During their Use by Consumers: Health Risk and Efficacy of Preservative Systems,” Letters in Applied Microbiology (2006), 43, pp. 301-306, A. VARVARESOU et al. “Self-Preserving in Cosmetics,” Int. J. Cosmet. Sci. (2009), 31(3), p. 175, A. DETMER et al. “A Guidance Document on Microbiological Control of Cosmetics Products” Danish Ministry of Environment. Environmental Project No. 1336, 2010).

Among these microbial agents that contaminate the cosmetic products, it is possible to cite bacteria (in particular those of the Staphylococcus type), molds (such as those of the Aspergillus type) and even sometimes spores (this is in particular the case of floral waters or plant extracts used as ingredients in cosmetic compositions).

The method can therefore be used to inactivate at least one pathogenic microorganism in the plant state or in the spore-forming state that is present in the cosmetic product.

The object of the invention is in particular the use of the method for treatment of a cosmetic product for the sterilization of cosmetic products and/or for improving their preservation.

The method makes possible a total and irreversible decontamination that is equivalent to cold sterilization but while preserving the inner structure of the cosmetic product and its organoleptic properties.

The specific combination of the parameters of the method according to the invention makes it possible to lead in a single step to a total inactivation of the microbial agents that contaminate the cosmetic product, be they plant species or spore-forming species. According to the nature of the microorganisms and the composition of the cosmetic product, the level can reach: 10⁹ (Destructive Effectiveness ED=9) for the plant species and greater than 10⁶ (ED>6) for the spore-forming, in particular bacterial, species.

According to another aspect, the treatment method according to the invention can also be used to modify the texture of cosmetic products. It is actually possible to associate a modification of the texture of the cosmetic product with the microbiological securing treatment by application of the method according to the invention if an ingredient or component (for example, a polysaccharide), able to gel in an irreversible manner under the conditions of said cosmetic product treatment method according to the invention, is introduced during the formulation.

Finally, the object of the invention is also a cosmetic product that is obtained by the implementation of the method, characterized by the fact that it does not contain a preserving agent and that it has an inactivation rate of pathogenic microorganisms that are present in the medium of at least 6 log (destructive effectiveness at least equal to 6) in relation to the initial product.

Inactivation rate according to the invention is defined as, for example:
An inactivation rate of 2 log, means that 99% of the pathogenic microorganisms that are present in the medium are inactivated,
An inactivation rate of 4 log, means that 99.99% of the pathogenic microorganisms that are present in the medium are inactivated,
A rate of 5 log, means than 99.999% of the pathogenic microorganisms that are present in the medium are inactivated,
A rate of 6 log, means that 99.9999% of pathogenic microorganisms present in the medium are inactivated.

Preferably, the cosmetic product does not contain preserving agents of any kind and no longer contains any pathogenic microorganism, and its properties and its structure are identical to those before treatment. As a result, the cosmetic product thus treated by the method retains its natural nature. This method seems most particularly suitable for the products that display the name “Bio.”

In addition, unlike a cosmetic product that is treated by high temperatures, such as, for example, a heat pasteurization that makes it possible to eliminate the pathogenic agents, the treated cosmetic product according to the invention is not denatured by heat and preserves its structure and its properties because the method according to the invention does not heat the cosmetic product to a high temperature.

Thus, through the method according to the invention, it is possible to obtain cosmetic products without any preservatives, bactericides and additives (designed either to inactivate the pathogenic microorganisms or to limit their development) and totally free of microbial contamination without altering the tight structure of these products and also their organoleptic properties. This treatment is therefore equivalent to cold sterilization.

The conditions of the method according to the invention can be optimized inside the ranges of values claimed based on the cosmetic product to be treated, of the nature of the pathogenic microorganisms that contaminate the product, and based on the desired use for obtaining the best possible inactivation rate. The conditions can also be optimized inside ranges of claimed values so that the total time of the treatment method is in agreement with the economic constraints suited to the treated medium.

The invention is now illustrated by nonlimiting examples and tests demonstrating the effectiveness of the method according to the invention.

For these examples, the effectiveness of the method according to the invention relative to the decontamination of a cosmetic product is evaluated by a certain number of parameters:
Nᵢ represents the initial concentration of microorganisms contaminating the cosmetic product and N_{f} represents the final concentration after application of the method according to the invention.
ED is the destructive effectiveness linked to the method

$$( E D = - log ( \frac{Nf}{Ni} ) ) .$$

### Example 1

Test of Decontamination of a Cosmetic Product without any Preservative and not Treated by a Microorganism Inactivation Technique.

The effectiveness of the method according to the invention was tested on a cream based on an emulsion and without any addition of preservatives. This cream, apart from water and minority components (on the order of 5 to 6%) had a significant concentration of various lipids (vegetable oil, etc.) of more than 15%. After its formulation and its manufacture, it was placed in a non-fluid-tight container and preserved at ambient temperature for approximately 4 days.

The initial contamination observed by microbiological analysis was on the order of 3·10⁴ UFC/g. This result demonstrated, on the one hand, the existence of an initial endogenic contamination either originating from ingredients used in the formulation or caused by handling, and, on the other hand, without any preservative, this contamination, at the end of several days, can reach a significant, and even large value, because of the microbial proliferation.

The various treatment parameters are provided in Table I below as well as the initial contamination (Nᵢ), the final contamination after HHP treatment (N_{f}), and the value of the destructive effectiveness (ED) for the endogenic flora.

TABLE I

| | HP Treatment Parameters | | | | Non-Inoculated | | |
|---|---|---|---|---|---|---|---|
| | VA | Ti | | P | “Formula 20” | | |
| | (MPa · s⁻¹) | (° C.) | MA | (MPa) | Nᵢ | N_{f} | ED |
| Test A | 3.3 | 20 | 12 minutes | 400 | >3.0 × 10⁴ | <1 | >4.5 |
| Test B | 3.3 | 20 | 3 × 4 minutes | 400 | <3.0 × 10⁴ | <1 | >4.5 |
| Test C | 3.3 | 36 | 3 × 4 minutes | 300 | 9.8 × 10⁵ | <1 | 6.0 |
| Test D | 3.3 | 36 | 3 × 4 minutes | 400 | 9.8 × 10⁵ | <1 | 6.0 |

It is observed that with a cyclic application mode or a continuous mode (3 cycles of 4 minutes or 12 minutes), the endogenic contamination is totally inactivated at a pressure of 400 MPa and a temperature of 20° C. or 36° C. By contrast, it was possible to observe that with a cyclic application mode and at a temperature of 20° C. (Test B), it was possible to inactivate the entire microbial feedstock from 300 MPa. This result indicates that the method according to the invention makes it possible to inactivate cosmetic products at moderate pressures and therefore in exchange to preserve particularly sensitive active ingredients (or components).

It is observed that the application of the method according to the invention under the conditions of Tests B and D leads to a total inactivation of the endogenic flora that contaminates the cosmetic product.

### Example 2

### Tests of Decontamination of the Same Cosmetic Product as the One of Example 1 but by Inoculating Doses of Various Microorganisms.

To evaluate the destructive effectiveness of the method according to the invention, two microorganisms have been selected for the challenge tests: a Gram+ bacterium (Staphylococcus aureus) and a mold (Aspergillus brasiliensis) that are often considered by the community of individuals in the cosmetics sector as common contaminants.

The same values of the parameters of the method according to the invention as those mentioned in Example 1 (Tests A to D) (See Table I) are applied.

TABLE II

| Inoculated with | | | Inoculated with | | |
|---|---|---|---|---|---|
| S. aureus | | | A. brasiliensis | | |
| Nᵢ | N_{f} | ED | Nᵢ | N_{f} | ED |
| Test A 1.7 × 10⁷ | 2.5 × 10⁰ | 6.8 | 1.6 × 10⁵ | <1 | 5.2 |
| Test B 1.7 × 10⁷ | <1 | 7.2 | 1.6 × 10⁵ | <1 | 5.2 |
| Test C 4.3 × 10⁴ | 5.0 × 10⁰ | 3.9 | 1.7 × 10⁵ | <1 | 5.2 |
| Test D 4.3 × 10⁴ | <1 | 4.6 | 1.7 × 10⁵ | <1 | 5.2 |

From the application of these tests that are representative of the method according to the invention, it is possible to conclude:
(i) That under the same treatment conditions, Aspergillus brasiliensis seems more sensitive to the application of the method than Staphylococcus aureus
(ii) That for totally inactivating Staphylococcus aureus under the conditions of this study, it is necessary to have a cyclic application (here, 3 cycles of 4 minutes and a pressure of 400 MPa, whether this is at 20° C. or at 36° C.) (cf. Tests B and D)
(iii) It is advantageous to emphasize that it is possible to reach ED values on the order of 7 for this Gram+ bacterium that is reputed to be very baroresistant in the prior art.

### Example 3

Test of the Method According to the Invention for Sterilization of Cosmetic Products on Another Cream that has Very Different Characteristics from Those Tested in Examples 1 and 2.

The tested cosmetic product is distinguished from the preceding one by the fact that no initial contamination (before any voluntary inoculation of microorganisms) has been observed.

The following table (Table III) provides treatment parameters according to the methods according to the invention that have been used.

TABLE III

| | VA | Ti | | | P |
|---|---|---|---|---|---|
| | (MPa · s⁻¹) | (° C.) | MA | | (MPa) |
| Test E | 3.3 | 20 | 12 | minutes | 400 |
| Test F | 3.3 | 20 | 3 × 4 | minutes | 400 |
| Test G | 3.3 | 36 | 3 × 4 | minutes | 300 |
| Test H | 3.3 | 36 | 3 × 4 | minutes | 400 |

This product was inoculated by doses of microorganisms (challenge tests). Two types of microorganisms have been used: a Gram+ bacterium (Staphylococcus aureus) and a mold (Aspergillus brasiliensis) that are considered by the community of the individuals in the cosmetics sector as common contaminants.

The destructive effectiveness of these microorganisms after application of the method according to the invention for decontamination of the cosmetic products (with the parameters of the method mentioned in Table III) is provided in Table IV.

TABLE IV

| HP | Inoculated with S. aureus | | | Inoculated with A. brasiliensis | | |
|---|---|---|---|---|---|---|
| Treatment | Nᵢ | N_{f} | ED | Nᵢ | N_{f} | ED |
| Test E | 1.7 × 10⁵ | 5.0 × 10⁰ | 4.5 | 1.1 × 10⁵ | <1 | 5.0 |
| Test F | 1.7 × 10⁵ | <1 | 5.2 | 1.1 × 10⁵ | <1 | 5.0 |
| Test G | 3.5 × 10⁵ | 2.5 × 10¹ | 4.1 | 9.0 × 10⁴ | <1 | 5.0 |
| Test H | 3.5 × 10⁵ | <1 | 5.5 | 9.0 × 10⁴ | <1 | 5.0 |

As for the preceding product (see Example 2), it is also noted that the mold-type microorganism (Aspergillus brasiliensis) is more sensitive to the effects of the method according to the invention than the Gram+ bacterium Staphylococcus aureus since regardless of the conditions, the inactivation is total.

By contrast, the conditions for the total inactivation of Staphylococcus aureus are more drastic, but they appear to be identical to those observed for the other cosmetic product (see Example 2).

It therefore seems possible, through the selection of the parameters of the method according to the invention, to be able to inactivate completely and in an irreversible manner the entire microbial feedstock (Aspergillus brasiliensis and Staphylococcus aureus). This total and irreversible inactivation is similar to cold sterilization.

### Example 4

The total decontamination of a cosmetic product by the method according to the invention could be applied industrially only if the tight structure of the product remained unaltered after application of such an HHP method.

In images that are representative of the cosmetic products, it was possible to observe a set of small bubbles characteristic of an emulsion of uniform size covering the entire surface under the microscope.

After treatment according to the conditions leading to the total inactivation of the inoculated pathogenic microorganisms (Aspergillus brasiliensis and Staphylococcus aureus), the observation with the microscope (magnification×500) shows bubbles of uniform size covering the entire surface of the plate of the microscope with a distribution that is identical to that of the control sample. It has been ensured that the tight structure of the cream (emulsion) has not been altered by the treatment according to the high hydrostatic pressure method. The organoleptic properties (color, odor, and visual aspect) are also preserved.

TABLE V
Experimental Conditions Leading to the Total
Inactivation of Aspergillus brasiliensis and
Staphylococcus aureus

| | VA | Ti | | P |
|---|---|---|---|---|
| | (MPa · s⁻¹) | (° C.) | MA | (MPa) |
| Test H | 3.3 | 36 | 3 × 4 minutes | 400 |

The implementation of the method according to the invention therefore clearly leads to a total inactivation of the contaminating microorganisms, without thereby altering the tight structure of the product or its organoleptic properties (color, odor, and visual aspect).

### Example 5

A cosmetic cream without any preservatives or additives that can cause a bactericidal effect or limit the growth of microorganisms was contaminated intentionally (challenge tests) by 4 microbial species either in the vegetative state (Staphylococcus aureus ATCC 6538, Enterobacter aerogeneses ATCC 13048) or in the spore-forming state (Aspergillus brasiliensis ATCC 16404).

The thus contaminated cream was subjected to two HHP treatments according to the invention (see Table VII).

TABLE VI
Experimental Conditions of the HHP A and HHP B Treatments.

| | VA (MPa/s) | Ti (° C.) | MA | P (MPa) | t_{latency} |
|---|---|---|---|---|---|
| HHP A | 1 | 35 | 4 cycles × | 350 | 5 minutes |
| | | | 5 minutes | | |
| HHP B | 3.3 | 35 | 20 minutes | 350 | |

The results that are obtained are presented in Table VII below:

TABLE VII
Evaluation of the Decontamination for Each of the
Microbial Species by Application of the HHP Treatments
According to the Invention

| | Initial | Contamination | |
|---|---|---|---|
| | Contamination | after HHP | Destructive |
| | Nᵢ | Treatment N_{f} | Effectiveness |
| Sample | (log.) | (log.) | ED |
| Cream + S. aureus, | 5.9 | | |
| Control | | | |
| Cream + S. aureus, | | 0 | 5.9 |
| HHP-A-Treated | | | |
| Cream + S. aureus, | | 0 | 5.9 |
| HHP-B-Treated | | | |
| Cream + A. brasiliensis, | 5.8 | | |
| Control | | | |
| Cream + A. brasiliensis, | | 0 | 5.8 |
| HHP-A- | | | |
| Treated | | | |
| Cream + A. brasiliensis, | | 0 | 5.8 |
| HHP-B- | | | |
| Treated | | | |
| Cream + E. aerogenese, | 5.5 | | |
| Control | | | |
| Cream + E. aerogenese, | | 0 | 5.5 |
| HHP-A- | | | |
| Treated | | | |
| Cream + E. aerogenese, | | 0 | 5.5 |
| HHP-B- | | | |
| Treated | | | |
| Cream + B. cereus, | 6.1 | | |
| Control | | | |
| Cream + B. cereus, | | 1 | 5.1 |
| HHP-A-Treated | | | |
| Cream + B. cereus, | | 2.9 | 3.2 |
| HHP-B-Treated | | | |

### Example 6

The same cosmetic cream as in Example 5, without any preservative or additive that can cause a bactericidal effect or limit the growth of microorganisms, has been intentionally contaminated (challenge tests) by 2 microbial species either in the vegetative state (Staphylococcus aureus ATCC 6538) or in the spore-forming state (Bacillus cereus ATCC14579).

The HHP treatment conditions of this cream are provided in Table VIII.

TABLE VIII
HHP Treatment Conditions.

| | VA (MPa/s) | Ti (° C.) | MA | P (MPa) | T_{latency} |
|---|---|---|---|---|---|
| HHP C | 1 | 35 | 2 cycles × 10 | 350 | 5 minutes |
| | | | minutes | | |
| HHP D | 6.6 | 35 | 20 minutes | 350 | |

The results of the microbiological analyses are provided in Table IX.

TABLE IX

| | Initial | | |
|---|---|---|---|
| | Contamination | Contamination after | Destructive |
| | Nᵢ | HHP Treatment N_{f} | Effectiveness |
| Sample | (log.) | (log.) | ED |
| Cream + S. aureus, | 9 | | |
| Control | | | |
| Cream + S. aureus, | | 0 | 9 |
| HHP-C-Treated | | | |
| Cream + S. aureus, | | 0 | 9 |
| HHP-D-Treated | | | |
| Cream + B. cereus, | 5.8 | | |
| Control | | | |
| Cream + B. cereus, | | 0.7 | 5.1 |
| HHP-C-Treated | | | |
| Cream + B. cereus, | | 2.9 | 2.9 |
| HHP-D-Treated | | | |

### Example 7

The same cosmetic cream as the one used for Examples 5 and 6, therefore without any additive, preservative or bactericide, has been inoculated, on the one hand, by the vegetative species Staphylococcus aureus with a very high initial contamination level, close to 10⁹ UFC/ml, and, on the other hand, by a spore-forming species Bacillus cereus with an initial level of contamination of close to 10⁶ UFC/ml.

This cream was placed in a flexible and deformable container so as to be certain of transmitting integrally to the product the characteristic parameters of the high hydrostatic pressure (HHP) method. This test container is then sealed so as to prevent any recontamination of the cream by the pressure-transmitting medium.

The characteristic parameters of the HHP treatment method are provided in Table X.

TABLE X

| | VA (MPa/s) | Ti (° C.) | MA | P (MPa) | Tᵢ (Minutes) |
|---|---|---|---|---|---|
| HHP E | 1 | 20 | 4 cycles of | 350 | 5 |
| | | | 5 minutes | | |

The time tc for application of pressure for each cycle is 5 minutes.

At the end of this treatment, the untreated control and the treated cream sample are used for inoculations in the media that are suitable for the growth of these microorganisms. After incubation, counts are then taken. The results of these microbiological analyses are provided in Table XI.

TABLE XI

| | Initial | | |
|---|---|---|---|
| | Contamination | Contamination after | Destructive |
| | Nᵢ | HHP Treatment N_{f} | Effectiveness |
| Sample | (UCF/ml) | (log.) | ED |
| Cream + S. aureus, | 7.2E+08 | 8.9 | |
| Control | | | |
| Cream + S. aureus, | 0 | 0 | 8.9 |
| HHP-E-Treated | | | |
| Cream + B. cereus, | 1.9E+06 | 6.3 | |
| Control | | | |
| Cream + B. cereus, | 9.5E+05 | 6.0 | 0.3 |
| HHP-C-Treated | | | |

1. A method for treating a cosmetic product by high hydrostatic pressure, comprising applying to the cosmetic product a pressure of between 200 MPa and 600 MPa with a continuous or cyclic application mode, wherein the pressure is applied at a compression rate of between 0.5 and 50 MPa/s.

2. The method for treating a cosmetic product according to claim 1, wherein the application mode is cyclic, and the pressure is released at a decompression rate between 1 and 50 MPa/s.

3. The method for treating a cosmetic product according to claim 1, wherein the total time for applying the pressure is between 2 and 60 minutes.

4. The method for treating a cosmetic product according to claim 1, wherein the initial temperature of the cosmetic product to be treated is between −10° C. and 40° C.

5. The method for treating a cosmetic product according to claim 1, wherein the application mode is cyclic and wherein the time for applying the pressure of each cycle is between 2 and 20 minutes.

6. The method for treating a cosmetic product according to claim 1, wherein the mode of application is cyclic and wherein at least two cycles have a time for applying the pressure that is different.

7. The method for treating a cosmetic product according to claim 1, wherein the application mode is cyclic and wherein for at least two cycles, the pressure in the cosmetic product is different.

8. The method for treating a cosmetic product according to claim 1, wherein the application mode is cyclic and wherein the pressure between two cycles is:
ambient pressure, or
an intermediate pressure between ambient pressure and a highest pressure applied during said two cycles.

9. The method for treating a cosmetic product according to claim 1, wherein the application mode is cyclic and wherein at least two cycles have a different compression rate and/or decompression rate.

10. The method for treating a cosmetic product according to claim 1, wherein the application mode is cyclic and wherein for at least one cycle, at least one of the compression rate or the decompression rate varies until reaching the pressure.

11. The method for treating a cosmetic product according to claim 1, wherein the application mode is cyclic and wherein at least two cycles have a different compression and/or decompression profile.

12. The method for treating a cosmetic product according to claim 1, wherein the application mode is cyclic and wherein for a cycle, the compression profile and the decompression profile are different.

13. The method for treating a cosmetic product according to claim 1, wherein the application mode is cyclic and wherein between at least two cycles, there is a latency time.

14. The method for treating a cosmetic product according to claim 13, wherein the application mode comprises at least three cycles and at least two different latency times.

15. The method for treating a cosmetic product according to claim 13, wherein the latency time is between 2 and 20 minutes.

16. The method for treating a cosmetic product according to claim 1, wherein said method is implemented in a high-pressure chamber that is closed by at least one seal.

17. The method for treating a cosmetic product according to claim 1, wherein the cosmetic product is contained in a final packaging or in a part of a final packaging for the implementation of the method.

18. The method for treating a cosmetic product according to claim 1, wherein the cosmetic product is a cosmetic composition designed to be applied on skin, mucous membranes, or integuments.

19. The method for treating a cosmetic product according to claim 1, wherein the cosmetic product is a cosmetic active ingredient or a cosmetic excipient designed to be integrated into a cosmetic composition.

20. A method for inactivating in a single treatment step at least one pathogenic microorganism in a vegetative state or in a spore-forming state that is present in a cosmetic product, comprising treating the cosmetic product by high hydrostatic pressure according to the method of claim 1.

21. A method for sterilizing a cosmetic product, comprising treating the cosmetic product by high hydrostatic pressure according to the method of claim 1.

22. A method for improving preservation of a cosmetic product, comprising treating the cosmetic product by high hydrostatic pressure according to the method of claim 1.

23. A method for texture modification of a cosmetic product, comprising:
introducing, during formulation of said cosmetic product prior to treatment by high hydrostatic pressure, a component that is able to gel in an irreversible manner under high hydrostatic pressure, and
treating the cosmetic product comprising said component by high hydrostatic pressure according to the method of claim 1.

24. A cosmetic product obtained by treating the cosmetic product by high hydrostatic pressure according to the method of claim 1, wherein said cosmetic product does not contain a preserving agent or bactericide, and wherein the cosmetic product has an inactivation rate of pathogenic microorganisms present in the cosmetic product that is from 6 log to 9 log in relation to the cosmetic product at an initial time.

25. A cosmetic product obtained by treating the cosmetic product by high hydrostatic pressure according to the method of claim 1, wherein properties and structure of the cosmetic product are identical before and after said treatment, wherein the cosmetic product does not contain a preservation agent or bactericide, and wherein the cosmetic product does not contain any pathogenic microorganism.

26. A device for implenting the method according to claim 1, comprising: a closed, flexible container (10), designed to accommodate a product (12), the flexible container (10) and the product (12) to be treated by high hydrostatic pressure,
an outer container (15) designed to accommodate said flexible container (10) with said product (12),
a distributor plug (17) provided with a canula (18) for intake, the canula being configured to perforate the lid (13) of said flexible envelope after treatment.

27. The device according to claim 26, wherein the outer container (15) comprises an air intake (19).

28. The device according to claim 26, wherein the flexible container is a flexible envelope that comprises a reservoir configured for receiving the product, a top with an opening (11), a lid (13) configured for hermetically sealing said reservoir of said envelope, and a head ring (14) configured for engaging with the outer container (15).
